(19) United States
(12) Patent Application Publication
(10) Pub. No.: US 2008/0311568 A1
(43) Pub. Date: Dec. 18, 2008

(54) Compositions and Methods for Nucleic Acid Analysis of Sequences with Insertions or Deletions

(75) Inventors: Phillip Kim, Irvine, CA (US); Vijay Mahant, Murrieta, CA (US)

Correspondence Address: FISH & ASSOCIATES, PC;ROBERT D. FISH, 2603 Main Street, Suite 1050, Irvine, CA, 92614-6232, US

(73) Assignee: AUTOGENOMICS, INC., Carlsbad, CA (US)

(21) Appl. No.: 11/721,641

(22) PCT Filed: Dec. 12, 2005
(86) PCT No.: PCT/US05/44899

§ 371 Date: Mar. 7, 2008

Related U.S. Application Data
(60) Provisional application No. 60/635,904, filed on Dec. 13, 2004.

(51) Int. Cl. C12Q 1/68; G01N 33/00; C07H 21/00

(52) U.S. Cl. 435/6; 436/94; 536/24.3

(57) ABSTRACT

Contemplated methods and kits include a plurality of single stranded oligonucleotides that comprise a discriminating sequence that is encompassed on one end by a label and on the other end by a unique tag sequence. In one preferred aspect, the discriminating sequence is an RNA repeat unit, forms a heteroduplex with a complementary DNA repeat unit of the target nucleic acid, and the discriminating agent is RNaseH. Using such systems, the number of repeat units can be simply identified by hydrolysis of the discriminating sequence where such sequence is adjacent to a predetermined number of DNA repeat units in the single stranded oligonucleotide.

This application claims priority to our copending U.S. provisional patent application with the Ser. No. 60/635,904, which was filed Dec. 13, 2004.

### FIELD OF THE INVENTION

The field of the invention is nucleic acid analysis, and especially analysis, detection, and/or determination of the presence and/or absence of bases (e.g., repeat units, insertions, deletions) within a known target sequence.

### BACKGROUND OF THE INVENTION

It is well established that mammalian and other genomes contain many DNA repeats, which are typically found throughout the chromatin and which vary in length from a single nucleotide to an entire gene. For example, mammalian highly repetitive DNA is often found in untranslated regions as a 6 to 10 base pair sequence at 10⁵ to 10⁶ copies, while full gene repeats, including histones, ribosomal RNA, tRNA, and SMN often exist as tandem clusters of multiple copies (e.g., 50 to 10⁴ copies). Still other DNA repeats are present as “hot spots” that may cause mismatching during DNA replication (e.g., Alu-repeats), that may form fragile chromosomal break points (e.g., CCG repeats). Furthermore, while the copy number of many repeats is often stable, various unstable repeats are also known (e.g., CAG repeats during DNA replication).

Depending on the particular size, distribution, and/or sequence characteristics, DNA repeat types may be classified into various groups, including (a) tandem repeats, which are often found in telomeres and are typically associated with various disease syndromes, (b) interspersed repetitive DNA, including short interspersed nuclear elements (e.g., Alu: GC rich, 280 bp length, or Mariner elements 80 bp, TA flanked), long interspersed nuclear elements (6-8 kb, variable sequence), (c) transposable elements with long terminal repeats (typically 1.5-10 kb, variable sequence), (d) DNA transposons (typically comprising two short inverted repeat sequences flanking the reading frame), and (e) trinucleotide repeats that often have various sizes, and tend to be associated with certain disease patterns.

Due to the mobile nature and/or sequence characteristics, DNA repeats may negatively interact with the genome by direct insertional mutagenesis (an estimate of 1 in 500 new germ line mutations is thought to be triggered by transposable elements), improper recombination between non-allelic repeats causing translocations and other re-arrangements, and presence of strong promotor regions that can cause inappropriate production of some proteins, and in some cases anti-sense RNA. For these and other reasons, numerous DNA repeats have been associated with certain diseases and syndromes (e.g., various types of spinocerebellar ataxia, Huntington's disease, schizophrenia, etc.). Moreover, as distribution, copy number, and/or length of DNA repeats are often highly variable from individual to individual, analysis of DNA repeats may also be used in forensic or other non-medical uses to correlate DNA-containing materials with DNA obtained from an individual with a relatively high degree of certainty.

To that end, numerous methods and tests have been developed to analyze DNA repeats. For example, DNA may be sequenced in a manual or automated manner to identify the type and length of a repeat. Such method advantageously allows identification of a repeat without specific knowledge of the particular sequence, however, is generally time consuming and often cost-ineffective. Alternatively, DNA repeats may be identified by RFLP analysis with direct hybridization and autoradiography using complementary probes or by PCR as taught in WO 93/16197. While RFLP and PCR methods are relatively fast and reliable, the exact number of repeats will typically not be determined.

In further known approaches, DNA repeats are employed as hybridization template for a guided ligation of oligos complementary to repeats as described in U.S. Pat. No. 5,695,933, EP 0 552 545, or EP 0 246 864. Similar to the methods described above, identification of repeats using such ligation methods is often fast and accurate. Unfortunately, such methods typically fail to provide exact determination of the copy number of the repeats. To overcome these and other disadvantages, discontinuous primer extension may be used as previously described in U.S. Pat. No. 5,945,284 or 6,309,829. Here, primer extension is carried out stepwise with detection and subsequent removal of the detectable label. Discontinuous methods generally allow for quantitative analysis of DNA repeats. However, most of such methods are relatively time consuming and tend to require operator attention.

More recently, advances in mass spectroscopy have significantly improved the range and accuracy in determining molecular weights of macromolecules, and thus allowed its application in the analysis of DNA repeats as taught in U.S. Pat. No. 6,764,822 or 6,090,558. While mass spectroscopy is accurate and fast, various disadvantages nevertheless remain. Among other things, mass spectroscopy is relatively expensive and typically requires a highly trained operator.

Thus, while numerous compositions and methods for nucleic acid analysis are known in the art, all or almost all of them, suffer from one or more disadvantages. Therefore, there is still a need for improved kits and methods for analysis of nucleic acids.

### SUMMARY OF THE INVENTION

The present invention is directed to compositions and methods of analysis, detection, and/or determination of the presence or absence of mutations, and particularly repeat units, insertions, and/or deletions within a known target sequence.

Most preferably, contemplated methods utilize a plurality of single stranded DNA oligonucleotides that include a variable number of repeat units complementary to the repeats in the DNA that is to be tested, wherein the last of the repeat units or a section towards the end of the repeat unit comprises RNA. Those oligos forming perfect hybrids (i.e., those with a number of repeat units equal or less than in the tested DNA) will be digested by RNaseH. Quantitation of the DNA repeats in the DNA to be tested in then performed by analysis of the single stranded DNA oligonucleotides after RNaseH digestion. Preferably, each of the oligos will hybridize on a predetermined position on a carrier and the RNaseH digest will result in a separation of a label from the remaining oligo.

In one aspect of the inventive subject matter, a test kit includes a plurality of first single stranded nucleic acids, each having a unique tag sequence coupled to a targeting sequence, and each further having a unique discriminating sequence coupled to the targeting sequence, wherein the discriminating sequence is further coupled to a label. In such compositions and methods, each of the unique discriminating sequences has a sequence suitable for at least partial hydrolysis of the single stranded nucleic acid by a discriminating agent under a discriminating condition to thereby separate the label from the unique tag sequence.

Most preferably, at least one of the single stranded nucleic acids is a chimeric molecule in which the targeting sequence comprises DNA and in which the discriminating sequence comprises RNA (with the discriminating sequence typically being the terminal repeat of the plurality of repeats), and the discriminating agent is RNaseH. In further preferred aspects, the label is an optically detectable label and especially suitable labels therefore include various fluorophors, luminophors, dyes, and an enzymes catalyzing conversion of a chromogen to a chromophore.

Where packages as a kit, it is generally contemplated that such kits will also comprise an instruction to (a) incubate a sample comprising a nucleic acid with the plurality of single stranded nucleic acids under the discriminating condition to form a test mixture, (b) apply the test mixture to a chip having a plurality of second single stranded nucleic acids, each of the second single stranded nucleic acids having a sequence complementary to the unique tag sequence and being located in a predetermined position, (c) acquire from the chip a plurality of signals from the labels, and (d) determine from the signals a genotype. Further additional components will therefore include a chip or other support having a plurality of second single stranded nucleic acids, wherein each of the second single stranded nucleic acids has a sequence that is complementary to the unique tag sequence and is located in a predetermined position or on an individually addressable solid phase (e.g., beads, strips, etc., using Raman spectroscopy). Where desirable, reagents may be included that comprise RNaseH, a polymerase, a terminal nucleotidyl transferase, and/or a labeled nucleotide.

Therefore, in another aspect of the inventive subject matter, a method of determining a genotype of a nucleic acid will include a step of incubating a sample nucleic acid with a plurality of first single stranded nucleic acids to thereby form a test mixture, wherein each of the first single stranded nucleic acids has a unique tag sequence that is coupled to a targeting sequence and further has a unique discriminating sequence that is coupled to the targeting sequence, wherein the discriminating sequence is further coupled to a label. In another step, a discriminating agent is added to the test mixture under discriminating conditions to thereby separate the label from at least one of the first single stranded nucleic acids, and in a still further step separation of the label from the first single stranded nucleic acids is determined using the unique tag sequence. In a still further step, the genotype is then determined from the step of determining.

Sample nucleic acids will preferably comprise an optionally methylated nucleic acid (e.g., amplicon, cDNA, genomic DNA, etc.). In further contemplated aspects, the unique tag sequence and the targeting sequence comprise a DNA while the discriminating sequence comprises a RNA. Therefore, particularly suitable discriminating conditions are conditions that allow hybridization of the sample nucleic acid with at least one, more typically at least some, and most typically all of the plurality of first single stranded nucleic acids. Further, it is generally preferred that the step of determining separation comprises binding the plurality of the first single stranded nucleic acids to a chip in predetermined positions using the unique tag sequence, and querying for a signal (e.g., illuminating with excitation light, or detection of reflected light) from the label in the predetermined positions.

In a still further contemplated aspect, a method of determining a copy number of a repeat unit in a nucleic acid has a step of combining a plurality of single stranded nucleic acids of the general formula A-T-Rₙ-Rₘ′-L with the nucleic acid under hybridization conditions to form a duplex, wherein A is a unique tag DNA sequence, T is a targeting DNA sequence, R is a DNA repeat sequence, n is an integer between 1 and 1000 (or even higher), inclusive, Rₘ′ is a RNA repeat sequence, m is an integer between 1 and 100 (or even higher), and L is a label. In another step, at least part of R′ is hydrolyzed using RNaseH in a duplex where R′ and the repeat unit in the nucleic acid form a complementary double strand to thereby separate L from A, and in yet another step the single stranded nucleic acids is immobilized onto a chip in predetermined positions using A. Then, a signal is measured from the predetermined positions. Typically, T has a length of at least 12 bases and has at least 90% complementarity with a portion of the nucleic acid. Such methods may further include a step of selectively labeling the single stranded nucleic acids in which R′ was at least partially hydrolyzed, wherein the step of labeling is performed using a second label that is distinguishable from L.

Consequently, chimeric oligonucleotides are contemplated having the general formula A-T-Rₙ-Rₘ′-L, wherein A is a unique tag DNA sequence, T is a targeting DNA sequence, R is a DNA repeat sequence, n is an integer between 1 and 1000 (or even higher), inclusive, Rₘ′ is a RNA repeat sequence, m is an integer between 1 and 100 (or even higher), and L is a label or any other discriminating factor.

Various objects, features, aspects and advantages of the present invention will become more apparent from the following detailed description of preferred embodiments of the invention.

### BRIEF DESCRIPTION OF THE DRAWING

FIG. 1 is a schematic illustration of a hybridization step in contemplated test compositions and methods.

FIG. 2 is a schematic illustration of a discrimination step in contemplated test compositions and methods.

FIG. 3 is a schematic illustration of an optional counter labeling step in contemplated test compositions and methods.

### DETAILED DESCRIPTION

The inventor has discovered that the presence and/or absence of bases (e.g., repeat units, insertions, deletions) within a known target sequence can be easily determined using a plurality of probe nucleic acids that have discriminating sequences of different lengths and hybridize to the target gene. In preferred assays, each of the probe nucleic acids includes a discriminating sequence of a specific length and a label, wherein presence or absence of the bases within the target gene is detected using a discriminating reaction implicating the discriminating sequence and the label on each of the probe nucleic acids.

For example, in one aspect of contemplated compositions and methods, a test kit has a plurality of single stranded nucleic acids, wherein each of the single stranded nucleic acids has a tag sequence on the 5′-end, which is followed by a targeting sequence (typically at least 90% complementary to a region that is adjacent to or includes the repeat elements in the DNA that is to be tested). Following the targeting sequence is a plurality of repeat elements that are at least partially complementary to the repeat elements in the DNA that is to be tested, wherein each of the plurality of single stranded nucleic acids has a distinct number of repeat elements. The last repeat element in the series of repeat elements is configured as a discriminating sequence. Depending on the particular choice and composition of the discriminating sequence, a label is either directly or indirectly coupled to the discriminating sequence. Most preferably, the discriminating sequence has a sequence suitable for at least partial hydrolysis of the single stranded nucleic acid by a discriminating agent under a discriminating condition to thereby separate the label from the unique tag sequence.

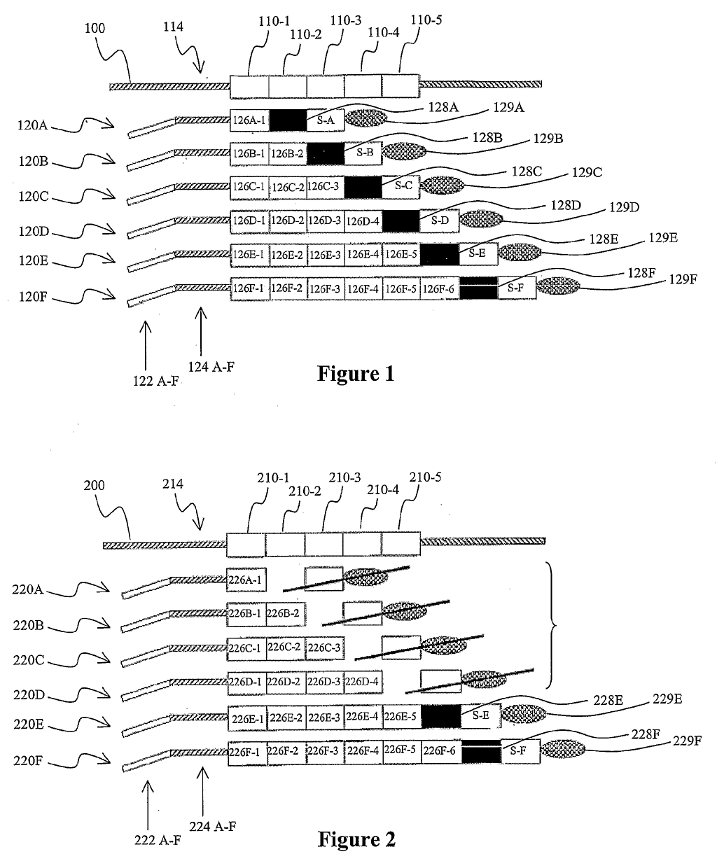

A particularly preferred assay is exemplarily illustrated in the attached Figures. Here, in FIG. 1 a target gene 100 with 5 repeats 110-1 to 110-5 is incubated with six distinct probe nucleic acids 120A to 120F. Typically, each of the probe nucleic acids 120A to 120F has an individual and distinct tag (122A to 122F) that corresponds to an anti-tag on a biochip (not shown), wherein the anti-tag is immobilized on the biochip in a predetermined position. Following the tag in each of the probe nucleic acids (in the direction of the 3′-end) is the sequence element (hatched, 124A to 124F) that is preferably entirely complementary to a portion of the target gene (114) to allow sequence specific hybridization under hybridization conditions. Following element 124A to 124F (in direction of the 3′-end) is a predetermined number of repeats 126A-1 to 126F-6, wherein each probe nucleic acid has a different number of repeats.

After the repeat section in each of the probe nucleic acids 120A to 120F is the discriminating sequence 128A to 128F, which represents the 3′-terminal repeat in the series of repeats, and which is comprised of RNA. In contrast, repeats 126A-1 to 126F-6 are typically comprised of DNA. Attached or coupled to the discriminating sequences 128A to 128F is a label 129A to 129F (via ssDNA spacer S-A to S-F, which may or may not be configured to further hybridize with the target DNA). Typically, the label is a fluorophor, and most preferably a 3′-terminal Cy3 label. Therefore, it should be recognized that the non-tag portion of the oligonucleotides will in most embodiments form a duplex with the gene to be analyzed so long as the number of repeats (including the discriminating repeat) is equal or less than the repeats found in the DNA to be analyzed. Once the last repeat fails to hybridize with the DNA to be analyzed, no duplex is formed. In FIG. 1, the duplex formed by 120A to 120D in the discriminating last repeat is a DNA-RNA heteroduplex. In contrast, the RNA portion in the discriminating last repeat of 120E and 120F has no complementary binding partner in the target DNA and will therefore be present as single stranded RNA.

After the probe nucleic acids have hybridized to the target sequence, discrimination is achieved in a discriminating reaction with a result as exemplarily depicted in FIG. 2. Here, RNAse H (not shown) was added to the hybrids (i.e., probe nucleic acids hybridized to the target gene), which resulted in digestion of those RNA portions that were hybridized to the corresponding portions of the target gene. Here, the RNA portion (discriminating sequence) of probes 220A to 220D was destroyed and consequently, the 3′-label and the spacer was removed from the 5′-portion (as represented by a strike through). In the remaining probe nucleic acids 220E and 220F in which the discriminating sequence had not hybridized, the label remained attached to the probe nucleic acid via the respective spacers. With respect to remaining numerals and components, the same considerations for items described in FIG. 1 apply to like items in FIG. 2.

Analysis of the reaction products can be carried out in this stage using numerous manners (see below). However, it is especially preferred that the probe-target nucleic acids are denatured or otherwise separated and that the probe nucleic acids are bound to the biochip via tag: anti-tag interaction. As each of the anti-tags on the chip is located at a predetermined position and binds only one specific probe nucleic acid, the pattern of the label can be used to determine the number of repeats. For example, unlabeled spots correspond to those probe nucleic acids which allowed for hybridization of the discriminating sequence to the target gene, while labeled spots on the biochip correspond to nucleic acids which did not allow for hybridization of the discriminating sequence to the target gene. Of course, it should be recognized that discrimination may also be performed on any alternative solid phase, which may or may not be immersed in a fluid. For example, where beads are employed, discrimination may be performed in a buffer on the beads. Similarly, discrimination may also be performed without hybridization to a solid phase using a real-time measurement. For example, FRET (fluorescence resonance energy transfer) labeling may be used to discriminate in real time.

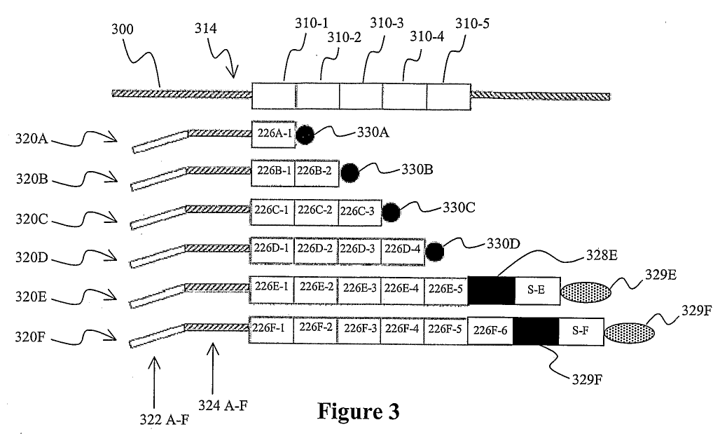

In further preferred aspects of the inventive subject matter, as exemplarily depicted in FIG. 3, counter-labeling can be used to further help discriminate signals. For example, and especially where the original probe nucleic acids had a 3′-terminal portion that was not suitable for enzymatic coupling of yet another labeled nucleotide (e.g., due to 2′,3′-dideoxy-nucleotide) counter-labeling can be achieved by a polymerase reaction that adds to the 3-OH groups of the hydrolysis products of the RNaseH reaction a detectable label (e.g., Cy5, represented by filled circle) using. Here, counter-labeling was achieved using a polymerase (not shown) to add labeled nucleotides 330A to 330D to the terminal 3-OH groups of probes 320A to 320D. Such reaction was not achieved in probes 320E and 320F as the 3′-ends of those probes lacked a OH group. With respect to remaining numerals and components, the same considerations for items described in FIG. 1 apply to like items in FIG. 3.

In alternative aspects of the inventive subject matter, it is contemplated that the probe nucleic acids may vary considerably, and that numerous modifications are deemed suitable for use herein. For example, it should be appreciated that while a unique tag for a particular probe is generally preferred, the tag need not necessarily be unique, nor even present. For example, the detection method following the discrimination reaction may be independent of positional placement, and especially contemplated detection methods include detection by mass difference. Among other suitable methods, mass spectroscopy, size exclusion chromatography, or gel electrophoresis may provide a clear identification of the products formed in the discrimination reaction. In another example, and especially where each probe has a unique label, the tag need not be unique, but may be one partner of an affinity pair. Typical representatives of such pairs include biotin-avidin/streptavidin, oligohistidine-NiNTA, etc. Thus, tags may be present or absent, unique, or identical, and may comprise a oligonucleotide and/or peptide. Especially preferred tags, however, will comprise a unique oligonucleotide sequence of between 5 to 100 nucleotides (e.g., at least 10, more typically at least 12, even more typically at least 15, and most typically at least 18 nucleotides), wherein the sequences are selected such that under a single hybridization condition each of the distinct tags will specifically hybridize with its complementary counterpart on the chip (and not with any sequence in the target DNA). Depending on the particular nature of the tag, it should be appreciated that the corresponding counterpart may be immobilized on a surface (e.g., oligo on a chip) or in a matrix material (e.g., agarose), or that the counterpart may be dissolved or suspended in a solution.

Most preferably, the tag is followed (directly or indirectly in direction of the 3′-end) by a single stranded DNA sequence element that is entirely or almost entirely complementary to the nucleotide sequence to which the probe will hybridize. Such sequence element is thought to increase specificity of the binding event to a predetermined region of the target gene to which the probe will hybridize, and optionally to align binding of the first repeat in the probe with the first repeat in the target DNA. The length of such sequence element is preferably at least between 8 and 12 nucleotides, more typically between 10 and 20 nucleotides, and most typically between 12 and 35 nucleotides. Furthermore, it is generally preferred that the sequence element is synthesized from DNA and contiguous with the tag element (which is also preferably synthesized from DNA). However, numerous alternative materials are also deemed suitable so long as such materials allow for specific hybridization to the target DNA. Among other contemplated materials, appropriate alternative sequence elements may include peptide nucleic acids, one or more modified nucleotides, etc.

Still further, it is contemplated that the sequence complementarity of the sequence element in the probe nucleic acid need not necessarily be perfect with the target gene. For example, suitable sequence elements may include one or more degenerate positions or modified nucleotide to allow for hybridization with mutated forms and/or SNPs within target gene. Alternatively, mismatches may be incorporated to adjust for expected mutations or even for annealing conditions where desirable. Therefore, it should be recognized that the sequence complementarity between the probe nucleic acids and the target DNA may also be less than 100%, and contemplated complementarities include those between 95% and 100%, between 90% and 100%, and even between 80% and 100%.

Similarly, it is generally preferred that the repeat and/or repeats in the probe nucleic acids are formed from single stranded DNA, wherein the repeats are contiguous with the sequence element and each other. However, where appropriate, spacer portions may be included between the sequence element and the first repeat unit. Furthermore, it is generally preferred that the repeat units are entirely complementary to the target repeats. In alternative aspects, however, it is also contemplated that the complementarities may also be lower (e.g., between 95% and 100%, between 90% and 100%, and even between 80% and 100%).

Depending on the particular nature and frequency of the repeat in the target DNA, it should be appreciated that the number of repeats in the probe nucleic acids may vary considerably. Thus, contemplated repeat numbers will typically be between one and several hundred (and even more), more typically between 1 and 100, and most typically between 1 and 50. Consequently, it should be appreciated that the kits and compositions according to the inventive subject matter may include between 1 and several hundred probe nucleic acids. However, where the number of repeats is relatively high, certain lengths may be omitted. For example, where a diseases is associated with repeats in excess of 30 repeats, contemplated kits and compositions may include probe nucleotides with 25 to 40 repeats. Moreover, the incremental increase of repeats need not be limited to single repeat increments, but may also be higher. For example, where relatively wide ranges of repeats are known for a gene, it is contemplated that kits and compositions may include probe nucleotides with 20, 30, 40, 50, etc. repeats. Alternatively, one or more repeats may also be replaced with a spacer, which may or may not be a nucleic acid. Suitable lengths of repeats are preferably between 1 and 100 nucleotides, more preferably between 1 and 50 nucleotides, and most preferably between 1 and 20 nucleotides. However, longer repeats are also considered suitable for use herein.

Therefore, and depending on the particular nature of the repeats, it should be noted that the probe nucleic acid may be synthetically prepared using solid phase synthesis, or in vitro using transcription of a suitable template. Further modifications may then be made in vitro by adding a tag, the discriminating sequence, or other portions.

With respect to the discriminating sequence, it is contemplated that the discriminating sequence represents at least part of one repeat, has a distinct physicochemical characteristic that will allow differentiation of hybridization to an at least partially complementary target DNA, and is preferably positioned at the 3′-end of the series of repeats. Most preferably, the discriminating sequence is entirely complementary to a repeat of the target DNA and has a sequence that spans over the entire repeat. However, in alternative aspects lower degrees of complementarity are also deemed suitable. Similarly, only part of the discriminating sequence may be configured to discriminate. Moreover, it should be recognized that the discriminating sequence need not be limited to a repeat sequence, but may also be a sequence that is complementary to a deletion or insertion known or suspected to be present in a target nucleic acid. Thus, it should be recognized that contemplated probe nucleic acids may also be employed to determine insertions, deletions, splice variations, etc.

In especially preferred aspects of the inventive subject matter, the discriminating sequence is at least in part made from RNA to thereby render a DNA:RNA heteroduplex formed between the probe nucleotide and the target DNA sensitive to RNaseH digestion. Alternatively, the discriminating sequence may also be rendered sensitive to digestion with a methylation sensitive restriction endonuclease (e.g., HpaII, MspI, HhaI, NotI, BclI, BspPI, Acc65I, Bme1390I, BseLI, BstXI, CfrI, etc). In such embodiments, the tag, the sequence element, and the repeats include methylation (e.g., partially or entirely overlapping dam or dcm methylation using 5-methylcytosine, N4-methylcytosine, 5-hydroxymethylcytosine, 5-hydroxymethyluracil, or N6-methyladenine) while the discriminating sequence (here: synthesized from ssDNA) is unmethylated. Therefore, only under conditions where the number of repeats in the probe nucleotide, including the discriminating sequence, is equal or less than the number of repeats in the target DNA restriction will occur at the discriminating sequence due to formation of a non-methylated restriction site. Similarly, and especially where the probe nucleotide is synthetically prepared on a solid phase, modified nucleotides may be employed in the repeats to render an otherwise sensitive restriction site insensitive to restriction. The last repeat will then include the corresponding non-modified nucleotide to thereby generate a sensitive restriction site.

Duplex formation of the discriminating sequence and the last repeat in the target DNA may be further stabilized by addition of a spacer that is at least partially complementary to the nucleic acid portion adjacent to the last repeat in the target DNA. Such spacer is typically between 1 and 50 nucleotides, more typically between 8 and 30 nucleotides and most typically between 12 and 18 nucleotides. While generally preferred, it is contemplated that the spacer may also be entirely omitted.

With respect to the label it is generally preferred that the label is covalently coupled to at least one of the discriminating sequence and the spacer, and that the label is radiometrically or optically or otherwise detectable in an automated fashion. Thus, and depending on the label and other considerations, the manner of detection may vary accordingly. However, it is generally preferred that the label is optically detected using automated detection of at least one of fluorescence, luminescence, and detection of a Raman-active moiety. Alternatively, detection may be performed using visual, scintigraphic, and/or radiographic detection. Still further, and especially where no label is present, detection of the reaction products of the discrimination reaction may be by size separation (e.g., using electrophoresis or mass spectroscopy), affinity separation etc. Where desired, the label may also be replaced with a signal generating moiety, and especially with an enzyme that converts a chromogenic substrate into a dye, or a luminogenic compound into a luminescent compound. Further contemplated labels include affinity markers that may then be visualized or otherwise detected.

Therefore, contemplated compounds especially include a chimeric oligonucleotide having the formula A-T-Rₙ-Rₘ′-L, wherein A is a unique tag DNA sequence, T is a targeting DNA sequence, R is a DNA repeat sequence, n is an integer between 1 and 1000, inclusive, Rₘ′ is a RNA repeat sequence (with a base sequence preferably identical/corresponding to R), n is an integer between 1 and 100, inclusive, and L is a label.

To confirm separation of the label from the tag at the 5′-end of the probe nucleic acid, counter staining may be employed. Most typically, such counter staining will rely on the possibility to modify the probe nucleic acid after the discriminating reaction. For example, the probe nucleic acid may be labeled with a secondary optically or otherwise detectable label that can only be attached to the probe nucleic acid where separation of the first label has taken place. There are numerous manner of labeling known in the art, and all of such manners are deemed suitable for use herein. However, particularly preferred manners include transfer of one or more fluorescently labeled nucleotides to the 3-′OH group of the probe nucleic acid. Such reaction may be rendered highly specific if the probe nucleic acid terminates before the discriminating reaction with a 2′,3′-dideoxynucleotide (to which no further nucleotide can be added). In less preferred aspects of the inventive subject matter, it is also contemplated that the spacer may be labeled with the label, and that the spacer includes a tag that will bind to an anti-tag in a predetermined manner such as to allow binding of that tag to a predetermined position. Detection of a signal from that position will then be indicative of a positive discrimination reaction (e.g., hydrolysis of discrimination sequence).

Alternatively, detection of the discriminating event (e.g., RNA hydrolysis, restriction, etc.) may be performed without hybridization of the single stranded nucleic acids to a solid phase, and in especially preferred alternative aspects, detection is carried out in real time in solution. For example, and especially where the discriminating event generates a 3′-hydroxy group at the 3′-terminus, detection can be carried out using real time PCR in test assays in which each distinct single stranded nucleic acid is disposed in a physically separate location (e.g., multi-well plate, reaction capillary, etc.). Here generation of the complementary strand can be measured using an intercalating dye. Alternatively, and especially where each of the discrimination reactions is performed in a separate location, FRET labels may be employed to identify the discriminating event.

In especially preferred aspects, contemplated kits and compositions will further include an instruction to incubate a sample comprising a nucleic acid with the plurality of single stranded nucleic acids under the discriminating condition to form a test mixture, and to apply the test mixture to a chip having a plurality of second single stranded nucleic acids, each of the second single stranded nucleic acids having a sequence complementary to the unique tag sequence and being located in a predetermined position. Such instructions will further provide advice to acquire from the chip a plurality of signals from the labels, and to determine from the signals a genotype (e.g., particular mutant, number of repeats, etc.).

Where desirable, contemplated kits may further comprise a chip having a plurality of second single stranded nucleic acids, wherein each of the second single stranded nucleic acids has a sequence that is complementary to the unique tag sequences and that is located in a predetermined position on the chip. Additionally, or optionally such kits may also have a reagent that includes RNaseH, a polymerase or terminal nucleotidyl transferase, and/or a labeled nucleotide.

Therefore, a method of determining a genotype of a nucleic acid will include a step in which a sample nucleic acid (e.g., optionally methylated amplicon, cDNA, or genomic DNA) is incubated with a plurality of first single stranded nucleic acids to thereby form a test mixture. In such methods it is generally preferred that each of the first single stranded nucleic acids has a unique tag sequence that is coupled to a targeting sequence and further has a discriminating sequence that is coupled to the targeting sequence, wherein the discriminating sequence is further coupled to a label. In another step, a discriminating agent is added to the test mixture under a discriminating condition (e.g., condition that allows hybridization of the sample nucleic acid with at least one and more typically all of the plurality of first single stranded nucleic acids) to thereby separate the label from at least one of the first single stranded nucleic acids, and in still another step, separation of the label from the at least one of the first single stranded nucleic acids is determined using the unique tag sequence. Finally, the genotype is deduced from the step of determining.

Consequently, and viewed from a different perspective, a method of determining a copy number of a repeat unit in a nucleic acid will include a step of combining a plurality of single stranded nucleic acids of the general formula A-T-Rn-R′-L with the nucleic acid under hybridization conditions to form a duplex, wherein A is a unique tag DNA sequence, T is an optional targeting DNA sequence, R is a DNA repeat sequence, n is an integer between 1 and 500 (or even more), inclusive, R′ is an RNA repeat sequence, and L is a label. In a further step, at least part of R′ is hydrolyzed using RNaseH in a duplex where R′ and the repeat unit in the nucleic acid form a complementary double strand to thereby separate L from A, and in yet another step, the single stranded nucleic acids are bound onto a chip in predetermined positions using A. In a still further step, a signal is measured from the predetermined positions. Most preferably, such methods include a step of selectively labeling the single stranded nucleic acids in which R′ was at least partially hydrolyzed, wherein the step of labeling is performed using a second label that is distinguishable from L. T has typically a length of at least 12 bases and has at least 90% complementarity with a portion of the nucleic acid.

Thus, specific embodiments and applications of nucleic acid analyses have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A test kit comprising:
a plurality of first single stranded nucleic acids, each having an unique tag sequence coupled to a targeting sequence, and each further having a discriminating sequence coupled to the targeting sequence, wherein the discriminating sequence is further coupled to a label; and
wherein each of the discriminating sequences has a sequence suitable for at least partial hydrolysis of the single stranded nucleic acid by a discriminating agent under a discriminating condition to thereby separate the label from the unique tag sequence.

2. The kit of claim 1 wherein at least one of the single stranded nucleic acids is a chimeric molecule in which the targeting sequence comprises DNA and in which the discriminating sequence comprises RNA.

3. The kit of claim 1 wherein the discriminating agent is RNaseH.

4. The kit of claim 1 wherein each of the discriminating sequences comprises a repeat sequence, and wherein each of the discriminating sequences has a distinct number of repeats.

5. The kit of claim 1 wherein the label is selected from the group consisting of a fluorophor, a luminophor, a dye, a Raman active moiety, and an enzyme.

6. The kit of claim 1 further comprising an instruction to (a) incubate a sample comprising a nucleic acid with the plurality of single stranded nucleic acids under the discriminating condition to form a test mixture, (b) apply the test mixture to a chip having a plurality of second single stranded nucleic acids, each of the second single stranded nucleic acids having a sequence complementary to the unique tag sequence and being located in a predetermined position, (c) acquire from the chip a plurality of signals from the labels, and (d) determine from the signals a genotype.

7. The kit of claim 1 further comprising a chip having a plurality of second single stranded nucleic acids, each of the second single stranded nucleic acids having a sequence complementary to the unique tag sequence and being located in a predetermined position.

8. The kit of claim 1 further comprising at least one of a reagent that includes RNaseH, a reagent that includes a polymerase or terminal nucleotidyl transferase, and a reagent that includes a labeled nucleotide.

9. A method of determining a genotype of a nucleic acid, comprising:
incubating a sample nucleic acid with a plurality of first single stranded nucleic acids to thereby form a test mixture;
wherein each of the first single stranded nucleic acids has a unique tag sequence that is coupled to a targeting sequence and further has a discriminating sequence that is coupled to the targeting sequence, wherein the discriminating sequence is further coupled to a label;
adding under a discriminating condition a discriminating agent to the test mixture to thereby separate the label from at least one of the first single stranded nucleic acids;
determining separation of the label from the at least one of the first single stranded nucleic acids using the unique tag sequence; and
deducing the genotype from the step of determining.

10. The method of claim 9 wherein the sample nucleic acid comprises an optionally methylated nucleic acid selected from the group consisting of an amplicon, a cDNA, and a genomic DNA.

11. The method of claim 9 wherein the unique tag sequence and the targeting sequence comprise a DNA and wherein the discriminating sequence comprises a RNA.

12. The method of claim 9 wherein the label is selected from the group consisting of a fluorophor, a luminophor, a dye, a Raman active moiety, and an enzyme.

13. The method of claim 9 wherein the discriminating condition is a condition that allows hybridization of the sample nucleic acid with all of the plurality of first single stranded nucleic acids.

14. The method of claim 9 wherein the discriminating agent is RNaseH or a methylation sensitive restriction endonuclease.

15. The method of claim 9 wherein the step of determining separation comprises binding the plurality of the first single stranded nucleic acids to a chip in predetermined positions using the unique tag sequence, and querying for a signal from the label in the predetermined positions.

16. A method of determining a copy number of a repeat unit in a nucleic acid, comprising:
combining a plurality of single stranded nucleic acids of the general formula A-T-Rₙ-R′-L with the nucleic acid under hybridization conditions to form a duplex;
wherein A is a unique tag DNA sequence, T is a targeting DNA sequence, R is a DNA repeat sequence, n is an integer between 1 and 500, inclusive, R′ is a RNA repeat sequence, and L is a label;
hydrolyzing at least part of R′ using RNaseH in a duplex where R′ and the repeat unit in the nucleic acid form a complementary double strand to thereby separate L from A;
binding the single stranded nucleic acids onto a chip in predetermined positions using A; and
measuring a signal from the predetermined positions.

17. The method of claim 16 further comprising a step of selectively labeling the single stranded nucleic acids in which R′ was at least partially hydrolyzed, wherein the step of labeling is performed using a second label that is distinguishable from L.

18. The method of claim 16 wherein T has a length of at least 12 bases and has at least 90% complementarity with a portion of the nucleic acid.

19. The method of claim 16 wherein the step of hydrolyzing is performed using RNaseH.

20. A chimeric oligonucleotide having the formula A-T-Rₙ-R′-L, wherein A is a unique tag DNA sequence, T is a targeting DNA sequence, R is a DNA repeat sequence, n is an integer between 1 and 500, inclusive, R′ is a RNA repeat sequence, and L is a label.

21. A method of determining a copy number of a repeat unit in a nucleic acid, comprising:
combining a plurality of single stranded nucleic acids of the general formula T-Rₙ-R′-L* with the nucleic acid under hybridization conditions to form a duplex;
wherein T is a targeting DNA sequence, R is a DNA repeat sequence, n is an integer between 1 and 500, inclusive, R′ is a RNA repeat sequence, and L* is an optional label, and wherein each of the single stranded nucleic acids has not a 3′-OH terminus;
hydrolyzing at least part of R′ using a discriminating agent when R′ and the repeat unit in the nucleic acid form a complementary double strand to thereby generate a 3′-OH terminus;
(a) adding at least one optionally labeled nucleotide to the 3′-OH terminus, or (b) adding a plurality of nucleotides to the 3′-OH terminus and labeling the added nucleotides; and
detecting the label in solution.

22. The method of claim 21 wherein the step of detecting is performed in real time.
